(19) United States
(12) Patent Application Publication
(10) Pub. No.: US 2011/0218532 A1
(43) Pub. Date: Sep. 8, 2011

(54) CORONOID FRACTURE FIXATION DEVICE

(76) Inventor: W. Andrew Eglseder, Owings Mills, MD (US)

(21) Appl. No.: 13/038,653

(22) Filed: Mar. 2, 2011

Related U.S. Application Data
(60) Provisional application No. 61/309,900, filed on Mar. 3, 2010.

(51) Int. Cl. A61B 17/56

(52) U.S. Cl. 606/64

(57) ABSTRACT

The invention provides a coronoid process fracture fixation device having an elongate body and a stop piece attached to one end. The elongate body is adapted for passing through the proximal ulna, across the fracture, and through the coronoid process. The stop piece, once it passes through the bone, deploys to an extended position approximately perpendicular to the elongate body and prevents the fixation device from backing out of the bone. Once inserted and the stop piece is deployed, the fixation device is fixed in place by a removable retaining device.

### CROSS REFERENCE TO RELATED APPLICATION

This application is based upon and claims benefit of copending and co-owned U.S. Provisional Patent Application Ser. No. 61/309,900 entitled “Coronoid Fracture Fixation Device”, filed with the U.S. Patent and Trademark Office on Mar. 3, 2010 by the inventor herein, the specification of which is incorporated herein by reference.

### BACKGROUND OF THE INVENTION

1. Field of the Invention

The present invention relates generally the treatment of fractures and to devices used for stabilizing bone fractures, and more particularly the invention relates to an umbrella/anchor for fixating fractures of a proximal ulna bone.

2. Description of the Background

The ulna is one of two long bones found in the human forearm. It is situated at the medial side of the forearm, parallel with the radius. Its upper extremity, of great thickness and strength, forms a large part of the elbow-joint; the bone diminishes in size from above downward, its lower extremity being very small. The ulna, like other bones, is susceptible to a variety of fractures and other dislocations. For example, fractures of the ulna are a common result of falls. In such falls, force exerted on the hands and wrist at impact frequently produces displacement of one or more bone fragments created distal to the fracture site. Fractures of the proximal ulna typically result from traumatic injuries such as sporting accidents and can be more frequent with age due to bone loss. Improper treatment can result in deformity and long-term discomfort. In particular, fractures of the coronoid process in the proximal ulna can lead to joint pain and elbow instability. Left untreated, the elbow joint of a patient can become unstable, stiff, and painful.

Fractures of the coronoid process, which is located on the proximal ulna, are typically small but difficult to treat. Coronoid process fractures are typically associated with posterior elbow dislocations. Generally, an isolated coronoid process fracture is rare; most will have associated radial fractures. Proper treatment is important since the coronoid fracture may have a heavy impact on overall elbow stability. Traditional fixation of these fractures involves capture of the coronoid fragments with screws or sutures coming from the posterior side of the ulna. This type of fixation may not be stable enough to resist the strong anterior dislocating force of the distal humerus.

There exists no similar device for fixation of the coronoid process. It would be desirable to provide a bone fixation device for use with the coronoid process of the proximal ulna. Moreover, a fixation device should provide desirable alignment and stabilization of the bone structure of the coronoid process. There remains a need for an improved device and method for repairing coronoid process fractures in such a manner that the likelihood of recreating or reproducing a pain-free and fully functional elbow joint is maximized.

### SUMMARY OF THE INVENTION

Accordingly, it is an object of the present invention to provide a device for fixation of a fracture of the coronoid process that avoids the disadvantages of the prior art.

It is an object of the present invention to provide a device for securing a coronoid process fragment to the remainder of a coronoid process of an ulna bone of a patient at a site of fracture during surgical repair of a coronoid process fracture.

It is another object of the present invention to provide a coronoid fracture fixation device having an elongate body and a deployable stop piece. A related object of the present invention is to provide a coronoid fracture fixation device configured and arranged so the deployable stop piece matches the shape and contour of a healthy unfractured coronoid process.

Another object of the present invention is to provide a coronoid fracture fixation device sized and configured to pass through the proximal ulna, across the fracture, and through the coronoid process.

These and other objects of the present invention are accomplished by providing a coronoid process fracture fixation device having an elongate body and a stop piece attached to one end. The elongate body is adapted for passing through the proximal ulna, across the fracture, and through the coronoid process. The stop piece, once it passes through the bone, deploys to an extended position approximately perpendicular to the elongate body and prevents the fixation device from backing out of the bone. Once inserted and the stop piece is deployed, the fixation device is set in place by a removable retaining device.

### BRIEF DESCRIPTION OF THE DRAWINGS

The above and other features, aspects, and advantages of the present invention are considered in more detail, in relation to the following description of embodiments thereof shown in the accompanying drawings, in which:

FIG. 1 is a schematic of the upper portion of a typical ulna bone with coronoid process.

FIG. 2 shows a typical ulna bone with a fracture of the coronoid process and a fixation device.

FIG. 3 shows an enlarged view of the upper portion the ulna bone in FIG. 2 with a fracture of the coronoid process and a fixation device.

FIG. 4 shows side view of the upper portion of the ulna bone in FIG. 3 having a fracture of the coronoid process and a fixation device.

### DETAILED DESCRIPTION OF EXEMPLARY EMBODIMENTS

The invention summarized above and defined by the enumerated claims may be better understood by referring to the following description, which should be read in conjunction with the accompanying drawings in which like reference numbers are used for like parts. This description of an embodiment, set out below to enable one to practice an implementation of the invention, is not intended to limit the preferred embodiment, but to serve as a particular example thereof. In addition, it is to be understood that the phraseology and terminology employed herein are for the purpose of description and should not be regarded as limiting. The invention is capable of other embodiments and of being practiced and carried out in various ways. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

The invention provides a device especially adapted for fixing fractures of the coronoid process. The fixation device is sized and configured to pass through the ulna and the coronoid process to assist in reduction of a complicated fracture.

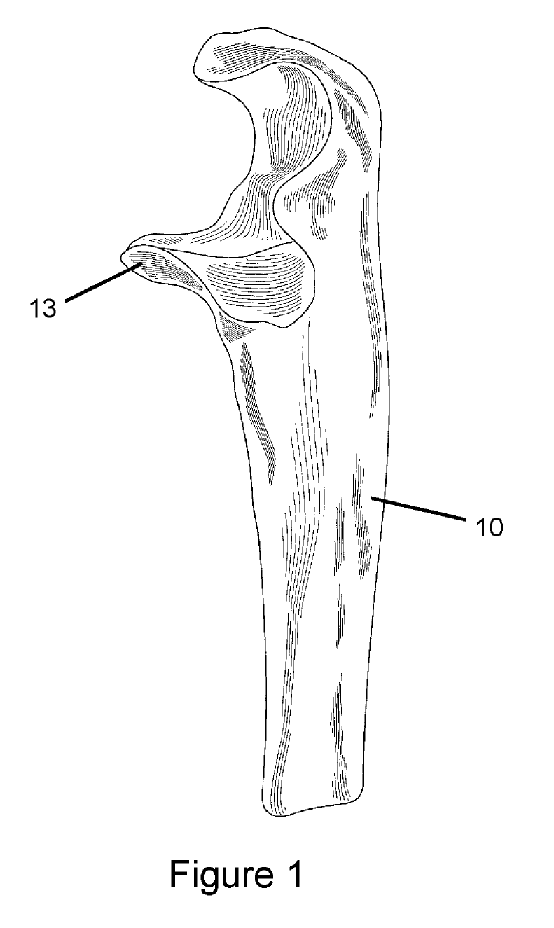

As illustrated in FIG. 1, the ulna 10 is broader proximally, and narrower distally. The coronoid process 13 is a triangular-like eminence projecting forward from the upper and front part of the ulna 10, which forms a portion of the elbow joint. The base of the coronoid process 13 is continuous with the body of the bone, and of considerable strength. Its apex is pointed, slightly curved upward, and, in flexion of the forearm, is received into the coronoid fossa of the humerus. Its upper surface is smooth and concave, and forms the lower part of the semilunar notch. Its antero-inferior surface is concave and marked by a rough impression for insertion of the brachialis.

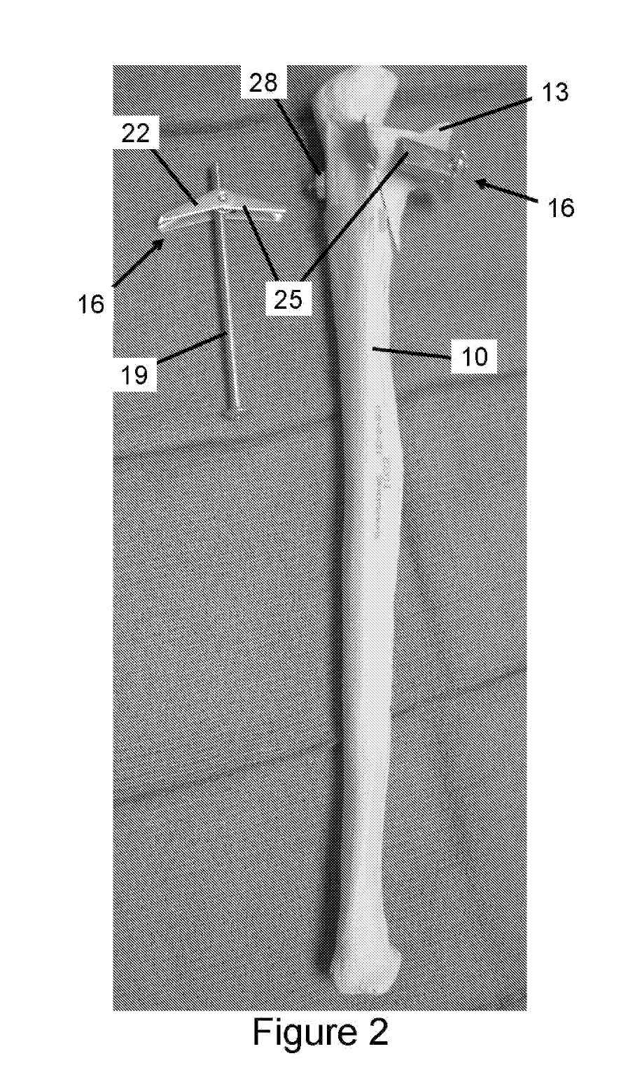

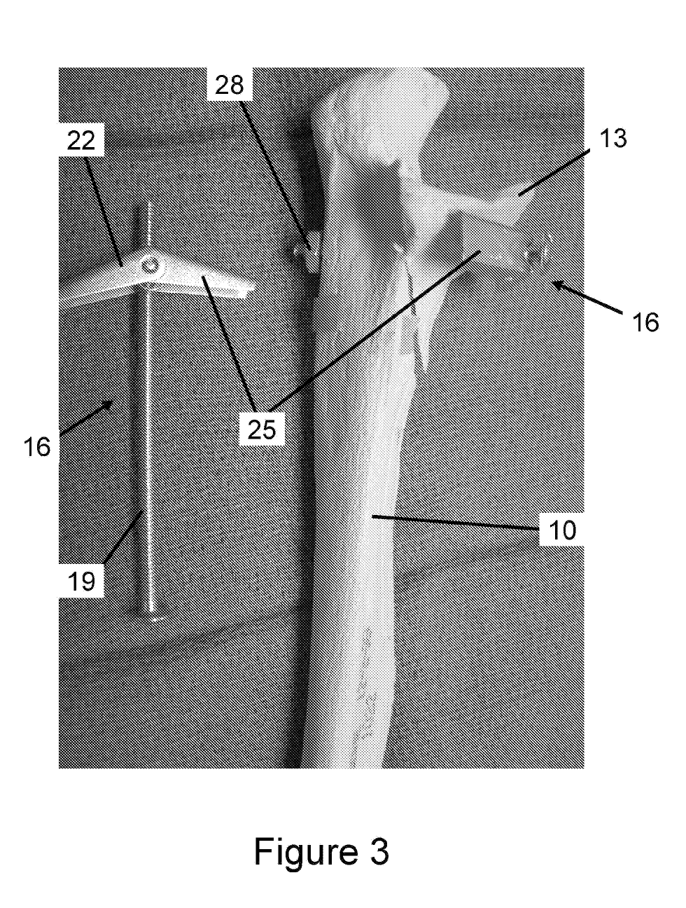

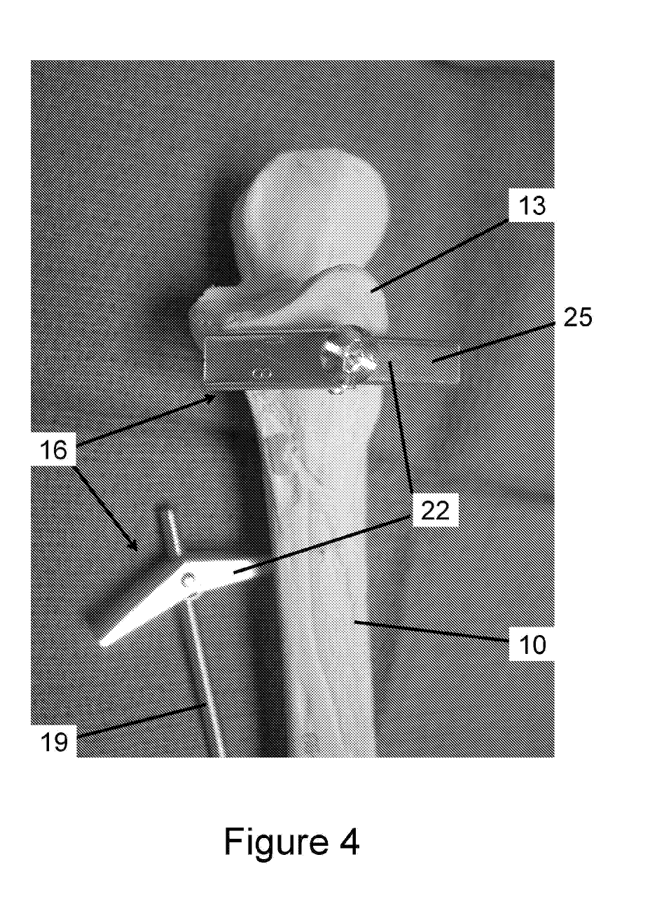

Referring to FIGS. 2-4, the fixation device of the present invention, indicated generally as 16, includes an elongate body 19 having a deployable stop piece 22 fixed on one end. Deployable stop pieces are generally described in U.S. Patent Application Publication Nos. 2010/0023010 and 2005/0182406, which are each incorporated herein by reference in their entirety. The elongate body 19 can be threaded or non-threaded along its length. In certain embodiments, the elongate body 19 is threaded along at least a portion of its length. The deployable stop piece 22 may be spring actuated or made of a resilient material that can be compressed against the elongate body 19 and, when released, is biased to extend away from the elongate body 19. The stop piece 22 may be approximately perpendicular to the elongate body 19 when extended away from the elongate body 19. In some embodiments, the deployable stop piece 22 may comprise one or more laterally extending fins 25. In some embodiments, the deployable stop piece 22 may comprise a single, substantially circular piece, somewhat like an umbrella. The elongate body 19 should be of a length at least sufficient to extend through the proximal ulna 10, across the fracture, and through the coronoid process 13. In certain embodiments, the elongate body 19 comprises a 3 mm bolt of a length at least sufficient to extend through the proximal ulna 10, across the fracture, and through the coronoid process 13. The length of the elongate body 19 may be adjustable or come in a fixed length that can be used by one of ordinary skill in the art considering the diameter of the ulna bone. For example, the elongate body 19 of a device of the present invention for use on a child or small person would be shorter than the elongate body 19 of a device of the present invention for use on a large adult.

In use, the fixation device 16 is used for stabilization of fractures of the coronoid process 13 of the ulna 10. The fracture would first be reduced and, at least temporarily, held with K-wire, or other method known by one of ordinary skill in the art. A drill, or other appropriate device, is used to make a hole through the proximal ulna 10, across the fracture, and through the coronoid process 13. The hole should be of sufficient diameter to enable the device 16 with a deployable stop piece 22 in an undeployed configuration, to pass therethrough. In certain embodiments, the fixation device 16 should be placed through the proximal ulna 10, across the fracture, and through the coronoid process 13 from a dorsal to an anterior direction. Typically, the device 16 is inserted through the hole sufficiently to allow the deployable stop piece 22 to extend outward and contact the coronoid process 13. According to the present invention, the deployable stop piece 22 prevents reverse motion of the fixation device 16 after it has been inserted. In certain embodiments, the deployable stop piece 22 may be appropriately configured to match the shape and contour of the coronoid process 13. After the fixation device 16 is inserted and the stop piece 22 has deployed, an appropriate retaining device, such as a nut 28, is applied to the remaining end of the elongate body 19 and tightened sufficiently. Any excess length of the elongate body 19 may be removed.

The invention has been described with references to specific embodiments. While particular values, relationships, materials and steps have been set forth for purposes of describing concepts of the invention, it will be appreciated by persons skilled in the art that numerous variations and/or modifications may be made to the invention as shown in the disclosed embodiments without departing from the spirit or scope of the basic concepts and operating principles of the invention as broadly described. It should be recognized that, in the light of the above teachings, those skilled in the art could modify those specifics without departing from the invention taught herein. Having now fully set forth certain embodiments and modifications of the concept underlying the present invention, various other embodiments as well as potential variations and modifications of the embodiments shown and described herein will obviously occur to those skilled in the art upon becoming familiar with such underlying concept. It is intended to include all such modifications, alternatives and other embodiments insofar as they come within the scope of the appended claims or equivalents thereof. It should be understood, therefore, that the invention may be practiced otherwise than as specifically set forth herein. Consequently, the present embodiments are to be considered in all respects as illustrative and not restrictive.

What is claimed is:

1. A coronoid fracture fixation device, comprising:
an elongate body having a first end and a second end; and
a deployable stop piece attached at a first end of the elongate body;
wherein said elongate body is sized and configured of a length at least sufficient to pass through a proximal ulna, across the fracture, and through the coronoid process of the proximal ulna.

2. The fracture fixation device according to claim 1 wherein said elongate body is threaded along at least a portion of its length.

3. The fracture fixation device according to claim 1, said deployable stop piece comprising at least one fin.

4. The fracture fixation device according to claim 3 wherein said at least one fin is approximately perpendicular to the elongate body when extended away from said elongate body.

5. The fracture fixation device according to claim 1, said deployable stop piece comprising a substantially circular umbrella.

6. The fracture fixation device according to claim 1 wherein said deployable stop piece is spring actuated to extend away from the elongate body.

7. The fracture fixation device according to claim 1 wherein said deployable stop piece comprises a resilient material biased to extend away from the elongate body.

8. The fracture fixation device according to claim 1 wherein said deployable stop piece is designed to match the shape and contour of the coronoid process.

9. The fracture fixation device according to claim 1, further comprising a retaining device removably attached to the second end of the elongate body.

10. A method of stabilizing a fracture of a coronoid process of the proximal ulna, comprising:
at least temporarily reducing the fracture and holding in place with K-wire, or the like;
making a hole through the ulna, across the fracture and through the coronoid process;
placing a fixation device into the hole through the ulna, across the fracture and through the coronoid process; and
attaching a retaining device to the fixation device.

11. The method according to claim 10 wherein said fixation device is placed through the ulna, across the fracture, and through the coronoid process from a dorsal to an anterior direction.

12. The method according to claim 11 wherein, after said fixation device is placed through the ulna, across the fracture, and through the coronoid process, a stop piece of the fixation device deploys to prevent the fixation device from moving from an anterior to a dorsal direction.

13. The method according to claim 10, further comprising the steps of:
sufficiently tightening the retaining device to hold the coronoid process in place.

14. The method according to claim 13, further comprising the steps of:
after said retaining device has been sufficiently tightened, removing any excess portion of said fixation device.

15. The method according to claim 10, said fixation device comprising:
an elongate body having a first end and a second end; and
a deployable stop piece attached at a first end of the elongate body;
wherein said elongate body is sized and configured of a length at least sufficient to pass through the proximal ulna, across the fracture, and through the coronoid process of the proximal ulna.

16. The method according to claim 15 wherein said elongate body is threaded along at least a portion of its length.

17. The method according to claim 15, said deployable stop piece comprising at least one fin.

18. The method according to claim 17 wherein said at least one fin is approximately perpendicular to the elongate body when extended away from said elongate body.

19. The method according to claim 15, said deployable stop piece comprising a substantially circular umbrella.

20. The method according to claim 15 wherein said deployable stop piece is spring actuated to extend away from the elongate body.

21. The method according to claim 15 wherein said deployable stop piece comprises a resilient material biased to extend away from the elongate body.

22. The method according to claim 15 wherein said deployable stop piece is designed to match the shape and contour of the coronoid process.
